(19) United States
(12) Patent Application Publication
(10) Pub. No.: US 2008/0138884 A1
(43) Pub. Date: Jun. 12, 2008

(54) Method for Separating and Purifying Nucleic Acid

(76) Inventors: Yumiko Takeshita, Saitama (JP); Hiroyuki Komazawa, Saitama (JP); Shinichi Watanabe, Saitama (JP)

Correspondence Address: BIRCH STEWART KOLASCH & BIRCH, PO BOX 747, FALLS CHURCH, VA, 22040-0747, US

(21) Appl. No.: 11/794,581

(22) PCT Filed: Mar. 2, 2006
(86) PCT No.: PCT/JP2006/304515

§ 371 Date: Jul. 2, 2007

(30) Foreign Application Priority Data
Mar. 4, 2005 (JP) 2005-060633

(51) Int. Cl. C07H 1/06; C12M 1/00

(52) U.S. Cl. 435/267; 435/283.1

(57) ABSTRACT

A method for separating and purifying nucleic acid, the method comprising: (1) a step of contacting a sample solution containing nucleic acid with a solid phase to adsorb the nucleic acid on the solid phase; (2) a step of contacting a washing solution with the solid phase to wash the solid phase in a state that the nucleic acid is adsorbed on the solid phase; and (3) a step of contacting a recovering solution with the solid phase to desorb the nucleic acid from the solid phase, wherein the sample solution is prepared by including a step of adding at least two enzymes.

### TECHNICAL FIELD

The present invention relates to a method for separating and purifying plasmid DNA widely used in a field of a recombinant nucleic acid technology.

### BACKGROUND ART

In many cases, nucleic acid is only obtained in an extremely small amount, and in addition to this, isolation and purification operations are complicated, and much time is required. Such a complicated operation often requiring much time tends to lead to loss of nucleic acid. The conventional method comprising incorporating the desired DNA into a coli plasmid vector, and culturing the same therein, thereby preparing a large amount of the desired DNA is one of the operations frequently conducted in a field of the recombinant nucleic acid technology. At the present stage, recovery of plasmid DNA from E. coli is carried out by removing genomic DNA, proteins and the like after a lytic operation to obtain a mixture of RNA and plasmid DNA by an alkali-SDS method or the like, and obtaining a purified plasmid DNA from the mixture of RNA and plasmid DNA generally using, for example, a column separation method using an ion-exchanged resin, or a cesium chloride density gradient super centrifugation. However, in the column separation using an ion-exchanged resin, the desired plasmid DNA is diluted with a large amount of an eluate. As a result, it is required to concentrate the plasmid DNA eluted, and this requires a complicated operation. Further, in the cesium chloride density gradient super centrifugation, a centrifugal treatment is conducted with a large-scaled apparatus at high number of revolution for a long period of time. As a result, the possible care should be taken, and further the treatment is complicated and is not economical.

On the other hand, a method for separating and purifying nucleic acid by adsorbing and desorbing nucleic acid on a solid phase comprising an organic polymer having a hydroxyl group on the surface thereof using a solution for adsorbing the nucleic acid on the solid phase and a solution for desorbing the nucleic acid from the solid phase respectively is reported as one of the method for simply separating and purifying the nucleic acid with good efficiency (JP-A-2003-128691).

### DISCLOSURE OF THE INVENTION

However, the technology proposed in JP-A-2003-128691 cannot sufficiently conduct removal of the included RNA in purification of plasmid from E. coli, and purity of the plasmid recovered is low. In view of the above, in conducting the method for separating and purifying the plasmid DNA from a solution containing RNA and plasmid DNA, it is desired for many samples to quickly recover the desired plasmid DNA in a high yield with high purity.

An object of the present invention is to provide a method for separating and purifying nucleic acid, comprising adsorbing a sample solution containing RNA and plasmid DNA on a surface of a solid phase, and desorbing the nucleic acid from the solid phase through washing or the like, thereby quickly obtaining high purity plasmid DNA in a high yield.

As a result of extensive investigations to overcome the problems in the conventional art, the present inventors have found that the desired nucleic acid (plasmid DNA) can be recovered quickly in a high yield with high purity by adsorbing a sample solution prepared by including a step of adding a plurality of different enzymes, i.e., a sample solution having a plurality of nucleases previously added thereto, on a solid phase. In particular, it has been ascertained in the present invention that yield and purity of the desired nucleic acid is markedly improved by using a solid phase comprising an organic polymer obtained by saponification of an acetylcellulose or a mixture of acetylcelluloses different each other in acetyl value. The present invention has been completed based on those findings.

According to the present invention, there is provided a method for separating and purifying the desired nucleic acid (plasmid DNA) by using a solution for adsorbing the desired nucleic acid in a sample solution containing the desired nucleic acid (plasmid DNA) prepared by the above preparation of the sample solution, and a solution for desorbing the nucleic acid from the solid phase, respectively.

The present invention achieves the above object by the following constitutions.

1. A method for separating and purifying nucleic acid, the method comprising:

(1) a step of contacting a sample solution containing nucleic acid with a solid phase to adsorb the nucleic acid on the solid phase;

(2) a step of contacting a washing solution with the solid phase to wash the solid phase in a state that the nucleic acid is adsorbed on the solid phase; and

(3) a step of contacting a recovering solution with the solid phase to desorb the nucleic acid from the solid phase,

wherein the sample solution is prepared by including a step of adding at least two enzymes.

2. The method for separating and purifying nucleic acid as described in 1 above,

wherein the at least two enzymes comprise at least one nucleic acid degrading enzyme.

3. The method for separating and purifying nucleic acid as described in 2 above,

wherein the at least one nucleic acid degrading enzyme is at least one RNA degrading enzyme.

4. The method for separating and purifying nucleic acid as described in 3 above,

wherein the at least one RNA degrading enzyme comprises RNase T1 and RNase A.

5. The method for separating and purifying nucleic acid as described in any of 1 to 4 above,

wherein the sample solution is prepared by adding a pre-treating solution containing at least one selected from a chaotropic salt, a surfactant, a defoaming agent, a nucleic acid stabilizer, a buffer, an acid and an alkali agent, and further adding a water-soluble organic solvent thereto.

6. The method for separating and purifying nucleic acid as described in any of 1 to 5 above,

wherein the solid phase is a membrane-shaped solid phase.

7. The method for separating and purifying nucleic acid as described in any of 1 to 6 above,

wherein the water-soluble organic solvent contains at least one selected from methanol, ethanol, propanol and its isomer and butanol and its isomer.

8. The method for separating and purifying nucleic acid as described in any of 1 to 7 above,

wherein the solid phase contains silica or its derivative, diatomaceous earth or alumina.

9. The method for separating and purifying nucleic acid as described in any of 1 to 8 above,

wherein the solid phase contains an organic polymer.

10. The method for separating and purifying nucleic acid as described in 9 above,

wherein the solid phase contains at least one selected from Teflon®, a polyester, a polyether sulfone, a polycarbonate, a polyacrylate copolymer, a polyurethane, a polybenzimidazole, a polyolefin, a polyvinyl chloride and a polyvinylidene fluoride.

11. The method for separating and purifying nucleic acid as described in 9 above,

wherein the solid phase contains nylon having positive or negative charges.

12. The method for separating and purifying nucleic acid as described in 9 above,

wherein the organic polymer has a polysaccharide structure.

13. The method for separating and purifying nucleic acid as described in 9 above,

wherein the organic polymer contains at least one selected from cellulose, cellulose mixed ester, cellulose nitrate, cellulose acetate and nitrocellulose.

14. An apparatus for automatically conducting the steps in a method for separating and purifying nucleic acid as described in any of 1 to 13 above.

15. A kit for conducting a method for separating and purifying nucleic acid as described in any of 1 to 13 above, the kit comprising:

(i) a cartridge for separation and purification of nucleic acid;

(ii) at least two enzymes;

(iii) a pre-treating solution containing at least one selected from a chaotropic salt, a surfactant, a defoaming agent, a nucleic acid stabilizer, a buffer, an acid and an alkali agent, and a water-soluble organic solvent;

(iv) a washing solution; and

(v) a reagent of a recovering solution.

### BRIEF DESCRIPTION OF THE DRAWING

FIG. 1 is a photograph obtained by electrophoresis of nucleic acid separated and purified according to the method of the present invention, and a molecular weight marker,

wherein M represents Molecular weight marker, Invitrogen 1 kb DNA Ladder; Q represents Extraction with QIAGEN QIAprep mini; 1 represents Adding only RNase A (a product of Wako Pure Chemical Industries, Ltd.) to a dispersing solution; 2 represents Adding only RNase T1 (a product of Sigma Co.) to a dispersing solution; 3 represents Adding RNase A (a product of Wako Pure Chemical Industries, Ltd.)+RNase T1 (a product of Sigma Co.) to a dispersing solution.

### BEST MODE FOR CARRYING OUT THE INVENTION

The method for separating and purifying nucleic acid according to the present invention is described in detail below.

### Preparation of Sample Solution

A sample containing nucleic acid, used in the present invention includes bacteria or cells.

The bacteria or cells that can be used are not particularly limited so long as those contain plasmid DNA.

In the present invention, the “nucleic acid” contained in the sample may be either of cyclic form and linear form, may be either of one chain and two chains, and may be either of DNA and RNA. The nucleic acid used does not have any limitation on its molecular weight. Further, the plasmid DNA as the desired nucleic acid may be either of double strand (ds) plasmid DNA and single strand (ss) phage DNA, and further does not have any limitation on its molecular weight.

The sample used herein means an optional sample containing nucleic acid. The kind of the nucleic acid in the sample may be one kind or a plurality of two or more kinds. The length of the individual nucleic acid is not particularly limited. For example, nucleic acid having optional length, such as from several bp to several Mbp, can be used. The length of nucleic acid is generally from several bp to several hundred kbp from the standpoint of handling property.

In the method of the present invention, the sample solution containing nucleic acid prepared from bacteria or cells is contacted with the solid phase to adsorb the nucleic acid in the sample solution on the solid phase, and the nucleic acid adsorbed on the solid phase is then desorbed from the sold phase.

As described above, in the method of the present invention, the sample solution is prepared by including a step of adding a plurality of different enzymes, and is adsorbed on the solid phase as a sample solution having a plurality of nucleases previously added thereto, whereby the desired nucleic acid (plasmid DNA) is separated and purified.

The sample solution is preferably prepared by adding a pre-treating solution containing at least one selected from a chaotropic salt, a surfactant, a defoaming agent, a nucleic acid stabilizer, a buffer, an acid and an alkali agent, and further adding a water-soluble organic solvent.

The sample solution is further preferably a solution prepared by:

dispersing bacteria or cells with a dispersing solution,

adding an alkali solution to dissolve bacteria or cells,

adding a neutralizing solution,

removing a precipitated component,

adding a lysis solution to a supernatant of the resulting precipitate, and

adding a water-soluble organic solvent.

The dispersing solution can contain at least one selected from a nucleic acid stabilizer and a buffer.

The alkali solution contains an alkali agent, and at least one selected from a surfactant, a defoaming agent and a buffer. Addition of the alkali solution enables bacteria or cells and nucleic acid in the solution dispersed with the dispersing solution to solubilize. As a result, a structure constituting cells is dissolved, and the nucleic acid can be dispersed in the sample solution. The alkali solution used includes an aqueous solution of an alkali metal analogue having a hydroxyl ion concentration of from 0.1 to 5 mol/liter. On the other hand, in place of the alkali solution, thermal modification can be used utilizing the properties that protein is weak to heat, but nucleic acid such as DNA is relatively strong to heat. Where the thermal modification is used, the heating conditions are preferably a heating temperature of from 80 to 100° C., and a heating time of from 5 to 20 minutes. Where the alkali solution is added, the thermal modification can be used alone or in combination thereof.

The neutralizing solution contains an acid, and at least one selected from a chaotropic salt, a surfactant, a defoaming agent, a nucleic acid stabilizer, a buffer, and an alkali metal analogue. The neutralizing solution functions to acidify an alkaline lysate obtained after adding the alkali solution, preferably by the addition of a mineral acid and an inorganic salt. The acid is preferably acetic acid. The acid concentration is not important for the present invention, and can be varied. The acid can use any inorganic salt so long as it dissolves in water. The preferable inorganic salt is salts in which its anion is the same as that of the acid. For example, where acetic acid is used, the mineral salt is preferably an alkali metal acetate or an alkaline earth metal acetate, particularly potassium acetate. The acid is used, which sufficiently lowers pH to an acidic region in a range of preferably from 4.0 to 6.5, and more preferably from 4.5 to 6.0. The salt concentration can also vary similar to the acid, but high salt concentration is preferable for the following reason. Where the solution obtained has high ion concentration, such a solution assists precipitation of chromosome DNA and other impurities, and this makes easy to separate plasmid DNA and those impurities. The salt concentration is most preferably in a range of from 1.0 to 10 mol/liter (based on monobasic salt).

The lysis solution contains at least one kind of compounds selected from a chaotropic salt, a surfactant, a defoaming agent, a nucleic acid stabilizer, a buffer, an alkali metal analogue, and a water-soluble organic solvent.

Desirably, undispersed cells do not remain in a solution obtained by dispersing with a dispersing solution. It is preferable that bacteria or cells are well dispersed with vortex, tapping, rollover mixing or the like, when dispersing the same.

### Enzyme

Addition period of a plurality of different enzymes can be any period. For example, those may be added to any of the above solutions. Further, besides the above solutions, those enzymes may be added at any time of preparation of the sample solution.

For example, RNA degrading enzyme solution can be added to a solution prepared by adding the lysis solution, thereby previously degrading unnecessary RNA.

Unnecessary DNA such as chromosome genome DNA can also be degraded by adding a specific DNA degrading enzyme solution to the solution obtained by adding the lysis solution. Further, unnecessary DNA such as chromosome genome DNA can be degraded by adding a specific DNA degrading enzyme solution to the solution containing the desired nucleic acid to be recovered.

Examples of the enzyme used in the present invention include protein degrading enzymes, nucleic acid degrading enzymes, and muramidase.

The protein degrading enzyme is not particularly limited, and for example, an alkali protease can preferably be used.

The nucleic acid degrading enzyme is not particularly limited, and for example, RNA degrading enzyme can preferably be used.

The RNA degrading enzyme is not particularly limited, and for example, RNase A or RNase T1 can preferably be used from the standpoints of enzyme activity and stability.

Examples of the DNA degrading enzyme that can preferably used include APT dependent exonuclease (trade name: Plasmid-Safe, a product of Epicenter Technologies, Madison, Wis., USA), and single-stranded specific endonuclease. Those enzymes specifically cut linear DNA (for example, genome DNA), but super coil-shaped plasmid NDA remains uncut.

The muramidase is not particularly limited, and for example, lysozyme can preferably be used.

A plurality of different enzymes are used in the present invention. The enzyme used is preferably nucleic acid degrading enzymes, and of those enzymes, RNA degrading enzyme is more preferable. As a plurality of different enzymes, RNase and RNase T1, which are RNA degrading enzyme, are particularly preferably used.

The respective enzyme can be used alone or as mixtures of its kind.

Concentration of the enzyme in the sample solution is preferably from 0.001 to 10 IU, and more preferably from 0.1 to 1 IU, per 1 ml of the total volume at the addition. Alternatively, the sample solution can be used in a concentration of from 0.05 to 20 mg/ml in terms of an action concentration.

A buffer can be added to the sample solution in order to stably maintain the action of enzyme. In this case, for example, Tris HCl can be added in an amount of from 1 to 200 mmol/liter.

Protein degrading enzyme not containing nucleic acid degrading enzyme, or muramidase can also preferably be used.

In addition, enzymes containing a stabilizer of protein degrading enzyme can preferably be used. The stabilizer than can preferably used is a metal ion. Examples of the metal ion include magnesium ion and calcium ion. Those ions can be added in the form of, for example, magnesium chloride and calcium acetate, respectively. Containing the stabilizer of protein degrading enzyme makes it possible to greatly decrease the amount of protein degrading enzyme necessary to recover nucleic acid, and as a result, the cost necessary to recover nucleic acid can be reduced. The solution of protein degrading enzyme can contain a buffer or a polyhydric alcohol. For example, Tris HCl can be added as the buffer in an amount of from 0.1 to 200 mmol/liter, or glycerol can be added as the polyhydric alcohol in an amount of from 1 to 70%. Those buffer and polyhydric alcohol can be used alone or as mixtures of two or more thereof, respectively.

### Surfactant

Examples of the surfactant used in the present invention include a nonionic surfactant, an anionic surfactant, a cationic surfactant and an ampholytic surfactant.

Of those, the anionic surfactant and nonionic surfactant can preferably be used.

Examples of the anionic surfactant include a sulfuric ester-based surfactant, a sulfonic acid-based surfactant, a carboxylic acid-based surfactant and a phosphoric acid-based surfactant. An alkylsulfuric ester salt is preferably used, and sodium dodecylsulate is more preferably used. The surfactant can preferably be contained in an alkali solution to act to dissolution of bacteria or cells.

Examples of the nonionic surfactant include a polyoxyethylene alkylphenyl ether-based surfactant, polyoxyethylene alkyl ether-based surfactant, and fatty acid alkanol amide. Of those, the polyoxyethylene alkyl ether-based surfactant is preferably used. Of the polyoxyethylene (hereinafter sometimes referred to as “POE” for brevity) alkyl ether-based surfactant, further preferable examples include POE decyl ether, POE lauryl ether, POE tridecyl ether, POE alkylene decyl ether, POE sorbitan monolaurate, POE sorbitan monooleate, POE sorbitan monostearate, tetraoleic acid polyoxyethylene sorbite, POE alkylamine, and POE acetylene glycol.

Those surfactants may be used alone or as mixtures of two or more thereof. Concentration of the surfactant in the alkali solution, neutralizing solution and lysis solution is preferably in a range of from 0.1 to 30% by mass. (In this specification, % by mass is equal to % by weight.)

### Buffer

Examples of the buffer that can be used in the present invention include pH buffers generally used. Preferable pH buffer is a biochemical pH buffer. Examples of the biochemical pH buffer include a buffer comprising a citric acid salt, a phosphoric acid salt or acetic acid salt, Tris-HCl, TE(Tris-HCl/EDTA), TBE (Tris-Borate/EDTA), TAE (Tris-Acetate/EDTA), and Good's buffer. Examples of the Good's buffer include MES(2-Morpholinoetahnesulfonic acid), Bis-Tris(Bis(2-hydroxyethyl)iminotris(hydromethyl)methane), HEPES(2-[4-[(2-Hydroxyethyl)-1-piperazinyl]etahnesulfonic acid, PIPES(Piperaxine-1,4-bis(2-ethanesulfonic acid)), ACES(N-(2-Acetamino)-2-aminoethanesulfonic acid), CAPS(N-Cyclohexyl-3-aminopropanesulfonic acid), and TES(N-Tris-(hydroxymethyl)methyl-2-aminoethanesulfonic acid).

Concentration of those buffers in the dispersing solution, alkali solution, neutralizing solution, lysis solution, washing solution and recovering solution is preferably in a range of from 1 to 300 mmol/liter.

### Nucleic Acid Stabilizer

Examples of the nucleic acid stabilizer used in the present invention include compounds having an action to inactivate activity of nuclease. Depending on the kind of the sample, for example, nuclease which degrades nucleic acid may be contained. In such as case, where nucleic acid is homogenized, the nuclease functions to nucleic acid, and as a result, yield of the nucleic acid may greatly be decreased. The nucleic acid stabilizer can make the nucleic acid in the sample be stably present, and this is preferable.

Compounds generally used as a reducing agent can be used as the nucleic acid stabilizer having an action to inactivate activity of nuclease. Examples of the reducing agent used include hydrogen; hydride compounds such as hydrogen iodide, hydrogen sulfide, lithium aluminum hydride or sodium borohydride; metals having large electropositive property, such as an alkali metal, magnesium, calcium, aluminum or zinc, or their amalgams; organic oxides such as aldehydes, saccharides, formic acid or oxalic acid; and mercapto compounds. Of those, the mercapto compounds are preferably used. Examples of the mercapto compound include N-acetylcysteine, mercaptoethanol and alkylmercaptan. In particular, β-mercaptoethanol is preferably used. The mercapto compound may be used alone or as mixtures of two or more thereof.

The nucleic acid stabilizer can be used in the treating solution in a concentration of preferably from 0.1 to 20% by mass, and more preferably from 0.3 to 15% by mass.

Chelating agent can also be used as the nucleic acid stabilizer having an action to inactivate activity of nuclease. Examples of the chelating agent that can be used include EDTA, NTA and EGTA. The chelating agent can be used alone or as mixtures of two or more thereof. For example, EDTA can be used in an action concentration range of from 1 to 300 mmol/liter. Preferably, the chelating agent can be contained in the dispersing solution to thereby act to inactivation of endogenous nuclease activity.

### Alkali Metal Analogue

Preferable examples of the alkali metal analogue used in the present invention include chlorides and acetylated products. Sodium salt, potassium salt and lithium salt are more preferable. The alkali metal analogue can be used in the dispersing solution, alkali solution, neutralizing solution, lysis solution, washing solution and recovering solution in a concentration of preferably 0.01 mol/liter or higher, and more preferably from 0.01 to 5 mol/liter.

### Defoaming Agent

Examples of the defoaming agent used in the present invention include a silicone-based defoaming agent (such as silicone oil, dimethyl polysiloxane, silicone emulsion, modified polysiloxane, or silicone compound), an alcohol-based defoaming agent (such as acetylene glycol, heptanol, ethyl hexanol, higher alcohol, or polyoxyalkylene glycol), an ether-based defoaming agent (such as heptyl cellosolve, or nonyl cellosolve-3-heptyl corbitol), an oils and fats-based defoaming agent (such as animal and plant oils), a fatty acid-based defoaming agent (such as stearic acid, oleic acid or palmitic acid), a metallic soap-based defoaming agent (such as aluminum stearate or calcium stearate), a fatty acid ester-based defoaming agent (such as natural wax or tributylphosphate), phosphoric ester-based defoaming agent (such as sodium octylphosphate), an amine-based defoaming agent (such as diamylamine), an amide-based defoaming agent (such as stearic acid amide), and other defoaming agents (such as ferric sulfate or bauxite). As the particularly preferable defoaming agent, two components of the silicone-based defoaming agent and the alcohol-based defoaming agent can be used in combination. Further, as the alcohol-based defoaming agent, an acetylene glycol-based surfactant is preferably used. The defoaming agent is used in the alkali solution, neutralizing solution, lysis solution and sample solution in a concentration of preferably from 0 to 10% by mass, and more preferably from 0.01 to 5% by mass.

### Chaotropic Salt

Examples of the chaotropic salt used in the present invention include a guanidine salt, sodium isocyanate, sodium iodide and potassium iodide. Of those, the guanidine salt is preferably used. Examples of the guanidine salt include guanidine hydrochloride, guanidine isothiocyanate and guanidine thiocyanate. Of those, guanidine hydrochloride is preferably used. Those salts can be used alone or as mixtures of two or more thereof. Concentration of the chaotropic salt in the neutralizing solution, lysis solution or sample solution is preferably 0.5 mol/liter or higher, more preferably from 0.5 to 4 mol/liter, and even more preferably from 1 to 3 mol/liter.

In place of the chaotropic salt, urea can be used as a chaotropic substance.

### Water-Soluble Organic Solvent

As described above, the sample solution is prepared by adding the pre-treating solution, and then adding the water-soluble organic solvent. The sample solution prepared by including the step of adding the water-soluble organic solvent is contacted with the solid phase. By this operation, nucleic acid in the sample solution is adsorbed on the solid phase. To adsorb nucleic acid solubilized by the above-described operation, it is necessary that the water-soluble organic solvent is mixed with a solubilized nucleic acid mixed solution, and further it is necessary that a salt is present in the sample solution obtained.

In other words, the nucleic acid is solublized in an unstable state by collapsing a hydration structure of water molecule present around the nucleic acid. When the nucleic acid in this state is contacted with the solid phase, it is considered that interaction proceeds between polar groups on the surface of nucleic acid and the surface of solid phase, preferably between polar groups on the surface of solid phase as described hereinafter, and as a result, the nucleic acid adsorbs on the surface of the solid phase. According to the method of the present invention, nucleic acid can be in an unstable state by that the water-soluble organic solvent is mixed with the solubilized nucleic acid mixed solution, and a salt is present in the sample solution obtained.

Examples of the water-soluble organic solvent include alcohols, acetone, acetonitrile, and dimethylformamide. Of those, alcohols are preferably used. The alcohols can be either of primary alcohols, secondary alcohols and tertiary alcohols. Of those, methanol, ethanol, propanol and its isomer, butanol and its isomer are preferably used.

The final concentration of the water-soluble organic solvent in the sample solution containing nucleic acid is preferably from 5 to 90% by mass, and more preferably from 20 to 60% by mass. It is particularly preferable that addition concentration of the water-soluble organic solvent is high as possible within an extent such that aggregates do not generate.

Preferable examples of the salt present in the sample solution obtained include various chaotropic substances (such as guanidium salt, sodium iodide or sodium perchlorate), sodium chloride, potassium chloride, ammonium chloride, sodium bromide, potassium bromide, calcium bromide, ammonium bromide, sodium acetate, potassium acetate and ammonium acetate.

The sample solution has pH of preferably from 3 to 10, more preferably from 4 to 9, and most preferably from 5 to 8.

The sample solution obtained preferably has a surface tension of 0.05 J/m² or lower, a viscosity of from 1 to 10,000 mPa, and a specific gravity of from 0.8 to 1.2. Preparing the solution having such physical properties has the following advantage. In the next step, after passing the sample solution containing nucleic acid through the solid phase and adsorbing the nucleic acid thereon, the residual solution can easily be removed.

### Solid Phase

The solid phase is preferably a solid phase on which nucleic acid adsorbs by the interaction to which ionic bond does not substantially participate. This means that “ionization” does not occur under the use conditions at the solid phase side, and it is presumed that the nucleic acid and the solid phase pulls against each other by changing environmental polarity. By this, nucleic acid can be isolated and purified with excellent separation performance and also good washing efficiency. This is presumed that the solid phase is a solid phase having hydrophilic groups, and the hydrophilic groups of nucleic acid and solid phase pull against each other by changing the environmental polarity.

The hydrophilic group used herein means a polar group (atomic group) that can possess interaction to water, and corresponds to all of groups (atomic groups) that participate in adsorption of nucleic acid. The hydrophilic group is preferably a group having a medium degree of strength of interaction to water (cf. CHEMICAL DICTIONAY, “Group having not so strong hydrophilicity” in Item “Hydrophilic Group”, published by Kyoritsu Shuppan Co.). Examples of such a hydrophilic group include hydroxyl group, carboxyl group, cyano group, and oxyethylene group. Of those, hydroxyl group is preferably used.

The solid phase having a hydrophilic group used herein means that a material itself forming a solid phase has a hydrophilic group, or a hydrophilic group is introduced into a material forming a solid phase by treatment or coating. Where the material forming a solid phase is subjected to treatment or coating, the material forming a solid phase may be either of organic materials and inorganic materials. Examples of the solid phase that can be used include a solid phase in which a material itself forming a solid phase is an organic material having a hydrophilic group, a solid phase in which a solid phase of an organic material having no hydrophilic group is treated to introduce a hydrophilic group, a solid phase in which a solid phase of an organic material having no hydrophilic group is coated with a material having a hydrophilic group to introduce a hydrophilic group, a solid phase in which a material itself forming a solid phase is an inorganic material having a hydrophilic group, a solid phase in which a solid phase of an inorganic material having no hydrophilic group is treated to introduce a hydrophilic group, and a solid phase in which a solid phase of an inorganic material having no hydrophilic group is coated with a material having a hydrophilic group to introduce a hydrophilic group. An organic material such as an organic polymer is preferably used as the material forming a solid phase from the standpoint of ease of processing.

The solid phase of a material having a hydrophilic group can include a solid phase of an organic material having a hydroxyl group. Examples of such a solid phase of an organic material having a hydroxyl group include polyhydroxyethyl acrylic acid, polyhydroxyethyl methacrylic acid, polyvinyl alcohol, polyvinyl pyrrolidone, polyacrylic acid, polymethacrylic acid, polyoxyethylene, polyamide (such as acryl-coated nylon; the nylon may be charged positively or negatively), polypropylene, and an organic polymer having a polysaccharide structure.

Examples of the organic polymer having a polysaccharide structure that can preferably be used include cellulose, hemicellulose, dextran, agarose, dextrin, amylose, amylopectin, starch, glycogen, pullulan, mannan, glucomannman, lichenan, isolichenan, laminaran, carrageenan, xylam, fructan, alginic acid, hyaronic acid, chondroitin, chitin, and chitosan. However, the organic polymer is not limited to the above materials so long as it has a polysaccharide structure and its derivative. Ester derivatives of the above any polysaccharide structure can also preferably be used. Further, saponified products of ester derivatives of the above any polysaccharide structure can preferably be used.

The ester of ester derivatives of the above any polysaccharide structure is preferably selected from at least one of carboxylic ester, nitric ester, sulfuric ester, sulfonic ester, phosphoric ester, phosphonic ester, and pyrophosphoric ester. Further, saponified products of carboxylic ester, nitric ester, sulfuric ester, sulfonic ester, phosphoric ester, phosphonic ester, and pyrophosphoric ester, of the above any polysaccharide structure can preferably be used.

The carboxylic ester of the above any polysaccharide structure is preferably selected from at least one of an alkylcarbonyl ester, an alkenylcarbonyl ester, an aromatic carbonyl ester, and an aromatic alkylcarbonyl ester. Further, saponified products of an alkylcarbonyl ester, an alkenylcarbonyl ester, an aromatic carbonyl ester, and an aromatic alkylcarbonyl ester of the above any polysaccharide structure can preferably be used.

The ester group of the alkylcarbonyl ester of the above any polysaccharide structure is preferably selected from at least one of acetyl group, propionyl group, butyroyl group, valer group, peptanoyl group, octanoyl group, decanoyl group, dodecanoyl group, tridecanoyl group, hexadecanoyl group, and octadecanoyl group. Further, saponified products of the above any polysaccharide structure having ester groups selected at least one of the acetyl group, propionyl group, butyroyl group, valer group, peptanoyl group, octanoyl group, decanoyl group, dodecanyl group, tridecanoyl group, hexadecanoyl group, and octadecanoyl group can preferably be used.

The ester group of the alkenylcarbonyl ester of the above any polysaccharide structure is preferably selected from at least one of acrylic group and methacrylic group. Further, saponified products of the above any polysaccharide structure having ester groups selected at least one of acrylic group and methacrylic group can preferably be used.

The ester group of the aromatic carbonyl ester of the above any polysaccharide structure is preferably selected from at least one of benzoyl group and naphthaloyl group. Further, saponified products of the above any polysaccharide structure having ester groups selected at least one of benzoyl group and naphthaloyl group can preferably be used.

Examples of the nitric acid ester of the above any polysaccharide structure that can preferably used include nitrocellulose, nitrohemicellulose, nitrodextran, nitroagarose, nitrodextrin, nitroamylose, nitroamylopectin, nitroglycogen, nitropullulan, nitromannan, nitroglucomannman, nitrolichenan, nitroisolichenan, nitrolaminaran, nitrocarrageenan, nitroxylam, nitrofructan, nitroalginic acid, nitrohyaronic acid, nitrochondroitin, nitrochitin, and nitrochitosan.

Further, saponified products of the above nitrocellulose, nitrohemicellulose, nitrodextran, nitroagarose, nitrodextrin, nitroamylose, nitroamylopectin, nitroglycogen, nitropullulan, nitromannan, nitroglucomannman, nitrolichenan, nitroisolichenan, nitrolaminaran, nitrocarrageenan, nitroxylam, nitrofructan, nitroalginic acid, nitrohyaronic acid, nitrochondroitin, nitrochitin, and nitrochitosan can preferably be used.

Examples of the sulfuric ester having the above any polysaccharide structure that can preferably be used include cellulose sulfate, hemicellulose sulfate, dextran sulfate, agarose sulfate, dextrin sulfate, amylose sulfate, amylopectin sulfate, glycogen sulfate, pullulan sulfate, mannan sulfate, glucomannman sulfate, lichenan sulfate, isolichenan sulfate, laminaran sulfate, carrageenan sulfate, xylem sulfate, fructan sulfate, alginic sulfate, hyaronic sulfate, chondroitin sulfate, chitin sulfate, and chitosan sulfate. Further, saponified products of the above cellulose sulfate, hemicellulose sulfate, dextran sulfate, agarose sulfate, dextrin sulfate, amylose sulfate, amylopectin sulfate, glycogen sulfate, pullulan sulfate, mannan sulfate, glucomannman sulfate, lichenan sulfate, isolichenan sulfate, laminaran sulfate, carrageenan sulfate, xylem sulfate, fructan sulfate, alginic acid sulfate, hyaronic acid sulfate, chondroitin sulfate, chitin sulfate, and chitosan sulfate can preferably be used.

The sulfonic ester having the above any polysaccharide structure is preferably selected from at least one of alkyl suflonic ester, alkenyl suflonic ester, aromatic suflonic ester, and aromatic alkyl suflonic ester. Further, saponified products of the above alkyl suflonic ester, alkenyl suflonic ester, aromatic suflonic ester, and aromatic alkyl suflonic ester can preferably be used.

Examples of the phosphoric ester having the above any polysaccharide structure that can preferably be used include cellulose phosphate, hemicellulose phosphate, dextran phosphate, agarose phosphate, dextrin phosphate, amylose phosphate, amylopectin phosphate, glycogen phosphate, pullulan phosphate, mannan phosphate, glucomannman phosphate, lichenan phosphate, isolichenan phosphate, laminaran phosphate, carrageenan phosphate, xylem phosphate, fructan phosphate, alginic phosphate, hyaronic phosphate, chondroitin phosphate, chitin phosphate, and chitosan phosphate. Further, saponified products of the above cellulose phosphate, hemicellulose phosphate, dextran phosphate, agarose phosphate, dextrin phosphate, amylose phosphate, amylopectin phosphate, glycogen phosphate, pullulan phosphate, mannan phosphate, glucomannman phosphate, lichenan phosphate, isolichenan phosphate, laminaran phosphate, carrageenan phosphate, xylem phosphate, fructan phosphate, alginic phosphate, hyaronic phosphate, chondroitin phosphate, chitin phosphate, and chitosan phosphate can preferably be used.

Examples of the phosphonic ester having the above any polysaccharide structure that can preferably be used include cellulose phosphonate, hemicellulose phosphonate, dextran phosphonate, agarose phosphonate, dextrin phosphonate, amylose phosphonate, amylopectin phosphonate, glycogen phosphonate, pullulan phosphonate, mannan phosphonate, glucomannman phosphonate, lichenan phosphonate, isolichenan phosphonate, laminaran phosphonate, carrageenan phosphonate, xylem phosphonate, fructan phosphonate, alginic phosphonate, hyaronic phosphonate, chondroitin phosphonate, chitin phosphonate, and chitosan phosphonate. Further, saponified products of the above cellulose phosphonate, hemicellulose phosphonate, dextran phosphonate, agarose phosphonate, dextrin phosphonate, amylose phosphonate, amylopectin phosphonate, glycogen phosphonate, pullulan phosphonate, mannan phosphonate, glucomannman phosphonate, lichenan phosphonate, isolichenan phosphonate, laminaran phosphonate, carrageenan phosphonate, xylem phosphonate, fructan phosphonate, alginic phosphonate, hyaronic phosphonate, chondroitin phosphonate, chitin phosphonate, and chitosan phosphonate can preferably be used.

Examples of the pyrophosphoric ester having the above any polysaccharide structure that can preferably be used include cellulose pyrophosphate, hemicellulose pyrophosphate, dextran pyrophosphate, agarose pyrophosphate, dextrin pyrophosphate, amylose pyrophosphate, amylopectin pyrophosphate, glycogen pyrophosphate, pullulan pyrophosphate, mannan pyrophosphate, glucomannman pyrophosphate, lichenan pyrophosphate, isolichenan pyrophosphate, laminaran pyrophosphate, carrageenan pyrophosphate, xylem pyrophosphate, fructan pyrophosphate, alginic pyrophosphate, hyaronic pyrophosphate, chondroitin pyrophosphate, chitin pyrophosphate, and chitosan pyrophosphate. Further, saponified products of the above cellulose pyrophosphate, hemicellulose pyrophosphate, dextran pyrophosphate, agarose pyrophosphate, dextrin pyrophosphate, amylose pyrophosphate, amylopectin pyrophosphate, glycogen pyrophosphate, pullulan pyrophosphate, mannan pyrophosphate, glucomannman pyrophosphate, lichenan pyrophosphate, isolichenan pyrophosphate, laminaran pyrophosphate, carrageenan pyrophosphate, xylem pyrophosphate, fructan pyrophosphate, alginic pyrophosphate, hyaronic pyrophosphate, chondroitin pyrophosphate, chitin pyrophosphate, and chitosan pyrophosphate can preferably be used.

Examples of the ether derivative having the above any polysaccharide structure that can be used include methyl cellulose, ethyl cellulose, carboxymethyl cellulose, carboxyethyl cellulose, carboxyethyl-carbamoylethyl cellulose, hydroxymethyl cellulose, hydroxyethyl cellulose, hydroxypropyl cellulose, hydroxypropyl methyl cellulose, hydroxyethyl methyl cellulose, cyanoethyl cellulose, and carbamoylethyl cellulose. However, the ether derivative is not limited to the above materials. Of those, hydroxymethyl cellulose and hydroxyethyl cellulose can preferably be used.

Compounds wherein hydroxyl groups in the above any polysaccharide structure are halogenated in an optional degree of substitution can also be preferably used.

The solid phase comprising the organic polymer having a polysaccharide structure that can preferably be used is acetylcellulose. Further, a solid phase of the organic polymer comprising a mixture of acetylcelluloses different from each other in acetyl value can also be used. Examples of the mixture of acetylcelluloses different from each other in acetyl value that can preferably be used include a mixture of triacetylcellulose and diacetylcellulose, a mixture of triacetylcellulose and monoacetylcellulose, a mixture of triacetylcellulose, diacetylcellulose and monoacetylcellulose, and a mixture of diacetylcellulose and monoacetylcellulose. Of those, a mixture of triacetylcellulose and diacetylcellulose can particularly preferably be used. The mixing ratio (mass ratio) of triacetylcellulose and diacetylcellulose is preferably 99:1 to 1:99, and more preferably 90:10 to 50:50.

Particularly preferable solid phase comprising acetylcellulose is a surface saponified product of acetylcellulose, as described in JP-A-2003-128691. The surface saponified product of acetylcellulose is a material obtained by saponification of a mixture of acetylcelluloses different from each other in acetyl value. A saponified product of a mixture of triacetylcellulose and diacetylcellulose, a saponified product of a mixture of triacetylcellulose and monoacetylcellulose, a saponified product of a mixture of triacetylcellulose, diacetylcellulose and monoacetylcellulose, and a saponified product of a mixture of diacetylcellulose and monoacetylcellulose can also preferably be used. A saponified product of a mixture of triacetylcellulose and diacetylcellulose is more preferably used. The mixing ratio (mass ratio) of triacetylcellulose and diacetylcellulose is preferably 99:1 to 1:99, and more preferably 90:10 to 50:50. In this case, the amount (density) of hydroxyl groups on the surface of solid phase can be controlled with the extent of saponification treatment (degree of saponification). A large amount (density) of hydroxyl groups is preferable in order to increase separation efficiency of nucleic acid. The degree of saponification of a solid phase obtained by saponification (degree of surface saponification) is preferably from 5 to 100%, and more preferably from 10 to 100%. Further, to increase a surface area of solid phase, it is preferable that a solid phase of acetylcellulose is subjected to saponification.

The saponification used herein means that acetylcellulose is contacted with a saponification solution (such as a sodium hydroxide aqueous solution). By this treatment, portion of the acetylcellulose contacted with the saponification solution converts into regenerated cellulose, resulting in introduction of hydroxyl groups. The regenerated cellulose thus prepared differs from the original cellulose in a crystal state and the like. It is particularly preferable in the present invention to use a solid phase of the regenerated cellulose as the solid phase.

To change the degree of saponification, saponification is conducted by changing the concentration of sodium hydroxide. The degree of saponification can easily be determined by NMR (for example, it can be determined by the extent of peak decrease of carbonyl groups).

A method for introducing a hydrophilic group into a solid phase of an organic material not having hydrophilic group is that a graft polymer chain having a hydrophilic group in a polymer chain or at a side chain is bonded to the solid phase.

There are two methods as the method of bonding the graft polymer chain to the solid phase of an organic material. One method is a method of chemically bonding the solid phase and the graft polymer chain, and another method is a method of polymerizing a compound having a polymerizable double bond using the solid phase as a starting point, thereby forming a graft polymer chain.

In the method of chemically bonding the solid phase and the graft polymer chain, a polymer having a functional group reacting with the solid phase at the terminal or side chain of the polymer is used. This functional group and the functional group of the solid phase are chemically reacted, thereby achieving grafting. The functional group reacting with the solid phase is not particularly limited so long as it can react with the functional group of the solid phase. Examples of the functional group reacting with the solid phase include a silane coupling group such as alkoxysilane, isocyanate group, amino group, hydroxyl group, carboxyl group, sulfonic group, phosphoric group, epoxy group, allyl group, methacroyl group, and acryloyl group.

Examples of the compound particularly useful as the polymer having a reactive functional group at the terminal or side chain of the polymer include a polymer having trialkoxysilyl group at the polymer terminal, a polymer having amino group at the polymer terminal, a polymer having carboxyl group at the polymer terminal, a polymer having epoxy group at the polymer terminal, and a polymer having isocyanate group at the polymer terminal. The polymer used in such an embodiment is not particularly limited so long as it has a hydrophilic group participating in adsorption of nucleic acid. Examples of such a polymer include polyhydroxyethyl acrylic acid, polyhydroxyethyl methacrylic acid and their salts; polyvinyl alcohol, polyvinyl pyrrolidone, polyacrylic acid, polymethacrylic acid and their salts; and polyoxyethylene.

The method of polymerizing a compound having a polymerizable double bond using the solid phase as a starting point, thereby forming a graft polymer chain is generally called a surface graft polymerization. The surface graft polymerization means a method of giving active species to a surface of a substrate by means of plasma irradiation, light irradiation, heating or the like, and bonding those to a sold phase by polymerization of a compound having a polymerizable double bond, which is arranged so as to contact with the solid phase.

It is necessary for the compound useful to form the graft polymer chain bonded to the substrate to be provided with two requirements of having a polymerizable double bond and having a hydrophilic group participating in adsorption of nucleic acid. Any compound of a polymer, an oligomer and a monomer, each having a hydrophilic group can be used as such a compound so long as it has a double bond in the molecule. Particularly useful compound is a monomer having a hydrophilic group.

Examples of the particularly useful monomer having a hydrophilic group are hydroxyl group-containing monomers such as 2-hydroxyethyl acrylate, 2-hydroxyethyl methacrylate, and glycerol monomethacrylate. Further, carboxyl group-containing monomers such as acrylic acid and methacrylic acid, or their alkali metal salts and amine salts can preferably be used.

Other method for introducing a hydrophilic group into a solid phase of an organic material not having hydrophilic group is that the solid phase is coated with a material having a hydrophilic group. The material used for coating is not particularly limited so long as it has a hydrophilic group participating in adsorption of nucleic acid. A polymer comprising an organic material is preferably used as the material used for coating from the standpoint of ease of working. Examples of such a polymer include polyhydroxyethyl acrylic acid, polyhydroxyethyl methacrylic acid and their salts; polyvinyl alcohol, polyvinyl pyrrolidone, polyacrylic acid and their salts; polyoxyethylene, acetylcellolose, and a mixture of acetylcelluloses different from each other in acetyl value. A polymer having a polysaccharide structure is preferably used.

A method can be employed that the solid phase of the organic material not having a hydrophilic group is coated with acetylcellulose or a mixture of acetylcelluloses different from each other in acetyl value, and the coating of acetylcellulose or a mixture of acetylcelluloses different from each other in acetyl value is subjected to saponification. In this method, the degree of saponification is preferably from 5 to 100%, and more preferably from 10 to 100%.

Examples of the solid phase which is an inorganic material having a hydrophilic group include solid phases containing silica or its derivative, diatomaceous earth, or alumina compound. The solid phase containing silica compound can be a glass filter. Further example of the solid phase can include a porous silica thin film as described in Japanese Patent 3,058,342. This porous silica thin film can be prepared by spreading a spreading solution of a cationic, amphipathic substance having a bimolecular film-forming ability on a substrate, removing a solvent from a solution film on the substrate to prepare a multilayer bimolecular thin film of the amphipathic substance, contacting the multilayer bimolecular thin film with a solution containing a silica compound, and extracting and removing the multilayer bimolecular thin film.

There are two methods as the method for introducing a hydrophilic group into the solid phase of an inorganic material not having hydrophilic group. One method is a method of chemically bonding the solid phase and a graft polymer chain having a hydrophilic group, and another method is a method of using a graft polymer chain using a monomer having a hydrophilic group having a double bond in the molecule thereof, and polymerizing the graft polymer chain using the solid phase as a starting point.

In the case of chemically bonding the solid phase and a graft polymer chain having a hydrophilic group, a functional group reacting with the functional group at the terminal of the graft chain is introduced into the inorganic material, and the graft polymer is then bonded thereto. Further, in the case of using a monomer having a hydrophilic group having a double bond in the molecule thereof, and polymerizing a graft polymer chain using the solid phase as a starting point, a functional group which acts as a starting point when polymerizing a compound having a double bond is introduced into the inorganic material.

The graft polymer having a hydrophilic group, and a monomer having a hydrophilic group having a double bond in the molecule can preferably use the graft polymer having a hydrophilic group, and a monomer having a hydrophilic group having a double bond in the molecule, as described in the method for introducing a hydrophilic group into the solid phase of the organic material not having hydrophilic group, respectively.

Another method for introducing a hydrophilic group into the solid phase of the inorganic material not having a hydrophilic group is that the solid phase is coated with a material having a hydrophilic group. The material used for coating is not particularly limited so long as it has a hydrophilic group participating in adsorption of nucleic acid. A polymer comprising an organic material is preferably used as the material used for coating from standpoint of ease of working. Examples of such a polymer include polyhydroxyethyl acrylic acid, polyhydroxyethyl methacrylic acid and their salts; polyvinyl alcohol, polyvinyl pyrrolidone and their salts; polyoxyethylene, acetylcellolose, and a mixture of acetylcelluloses different from each other in acetyl value.

A method can be employed that the solid phase of the organic material not having a hydrophilic group is coated with acetylcellulose or a mixture of acetylcelluloses different from each other in acetyl value, and the coating of acetylcellulose or a mixture of acetylcelluloses different from each other in acetyl value is subjected to saponification. In this method, the degree of saponification is preferably from 5 to 100%, and more preferably from 10 to 100%.

Examples of the solid phase comprising the organic material not having a hydrophilic group include metals such as aluminum; glasses; cements; ceramics such as china and porcelain; and solid phases prepared by processing new ceramics, silicon, activated carbon and the like.

The solid phase is preferably used in the form of a filter or a membrane for the reason that a solution can pass through the inside thereof. In this case, the solid phase has a thickness of preferably from 10 to 500 μm, and more preferably from 50 to 250 μm. The solid phase having small thickness as possible is preferable from the point of ease of washing.

The solid phase through which a solution can pass has an average pore size of preferably from 0.1 to 10 μm, and more preferably from 1 to 5 μm. By having such an average pore size range, a surface area sufficient to adsorb nucleic acid is obtained, and clogging is difficult to occur. The average pore size of the solid phase through which a solution can pass can be determined using a bubble point method (according to ASTM 316-86, and JIS 3832).

The solid phase through which a solution can pass may be a porous membrane having a front surface and a back surface symmetrical with each other, but a porous membrane having a front surface and a back surface asymmetrical with each other can preferably be used. The “front surface and back surface asymmetrical with each other” used herein means the property that physical properties or chemical properties of a porous membrane change from one side of the porous membrane to other side thereof.

One example of the physical properties of the membrane is an average pore size, and one example of the chemical properties is a degree of saponification.

When the porous membrane having a front surface and a back surface asymmetrical with each other is used in the present invention, it is preferable that the average pore size changes from “large average pore size” to “small average pore size” in the direction of passing a solution. Further, it is preferable to use a porous membrane having a ratio of the maximum pore size to the minimum pore size of 2 or higher. More preferably, the ratio of the maximum pore size to the minimum pore size is 5 or higher. By having such a ratio, a surface area sufficient to adsorb nucleic acid is obtained, and clogging is difficult to occur.

The solid phase through which a solution can pass has a porosity of preferably from 50 to 95%, and more preferably from 65 to 80%. Further, the bubble point is preferably from 0.1 to 10 kgf/cm², and more preferably from 0.2 to 4 kgf/cm².

The solid phase through which a solution can pass has a pressure loss of preferably from 0.1 to 100 kPa. By having such a pressure loss, uniform pressure is obtained when pressuring. More preferably, the pressure loss is from 0.5 to 50 kPa. The “pressure loss” used herein means the minimum pressure necessary to pass water through a membrane per 100 μm of the membrane thickness.

The solid phase through which a solution can pass has an amount of permeating water when passing water through the solid phase at 25° C. under a pressure of 1 kg/cm² of preferably from 1 to 5,000 ml, and more preferably from 5 to 1,000 ml, per 1 cm² of a membrane for 1 minute.

The solid phase through which a solution can pass has an adsorption amount of nucleic acid of preferably 0.1 μg or more, and more preferably 0.9 μg or more, per 1 mg of a porous membrane.

Flow rate when passing a sample solution containing nucleic acid through the solid phase is preferably from 2 to 1,500 μl/sec per area (cm²) of the solid phase in order to obtain an appropriate contact time of a solution to the solid phase. Where the contact time of a solution to the solid phase is too short, a sufficient separation and purification effect is not obtained. On the other hand, too long contact time is not preferable from the point of operating property. Further, the flow rate is preferably from 5 to 700 μl/sec per area (cm²) of the solid phase.

When a solution used can pass through the inside of the solid phase, the solution may be one kind of solution, but a plurality of solutions can be used. A plurality of solid phases may comprise the same or different material.

### Washing

After adsorbing nucleic acid on the solid phase, the solid phase is washed, whereby the recovery amount and purity of the nucleic acid are improved, and the amount of a sample containing the necessary nucleic acid can be reduced to a slight amount. Further, by automatically conducting washing and recovery operation, it is possible to conduct the operation simply and quickly. The washing step may be one washing for quick operation. Where purity of nucleic acid is further important, it is preferable that the washing is conducted repeatedly.

The washing solution is preferably a solution containing a water-soluble organic solvent. If desired and necessary, the washing solution may further contain a water-soluble salt, a buffer and a surfactant. The washing is required to have a function to wash out impurities in the sample solution adsorbed together with nucleic acid on the solid phase. To achieve this requirement, the washing solution is required to have a composition that nucleic acid does not desorb from the solid phase, but impurities desorb form the solid phase. The reason for this is that because nucleic acid is sparingly soluble in a water-soluble organic solvent such as an alcohol, such a composition is suitable to desorb components other than nucleic acid while holding the nucleic acid. Further, addition of the water-soluble salt increases adsorption effect of nucleic acid, and as a result, selective removal action of impurities and unnecessary components may be improved.

Examples of the water-soluble organic solvent contained in the washing solution include an alcohol and acetone. An alcohol is preferably used. Examples of the alcohol include methanol, ethanol, propanol, and butanol. The propanol may be either of isopronol and n-propanol, and the butanol may be either of linear butanol and branched butanol. Those alcohols can be used as mixtures of two or more thereof. Of those alcohols, ethanol is preferably used. The amount of the water-soluble organic solvent contained in the washing solution is preferably from 20 to 100% by mass, and more preferably from 40 to 100% by mass.

When the water-soluble salt is contained in the washing water, salts of halides are preferable. Of those, a chloride is more preferable. The water-soluble salt is preferably a monovalent or divalent cation. Alkali metal salts and alkaline earth metal salts are preferable. Of those, sodium salts and potassium salts are more preferable, and sodium salts are most preferable.

When the water-soluble salt is contained in the washing solution, the concentration of the water-soluble salt is preferably 10 mmol/liter or higher. The upper limit of the concentration is not particularly limited so long as it is in a range not impairing the solubility of impurities. However, the upper limit is preferably 1 mol/liter, and more preferably 0.1 mol/liter. The preferred embodiment is that the water-soluble salt is sodium chloride, and its concentration is 20 mmol/liter or higher.

The washing solution may not contain a chaotropic substance. Such a case can reduce the possibility of inclusion of the chaotropic substance in a recovering step subsequent to the washing step. Where the washing solution contains the chaotropic substance in the recovering step, the chaotropic substance frequently disturbs an enzyme reaction such as PCR reaction. Therefore, considering the subsequent enzyme reaction or the like, it is desirable that the washing solution does not contain the chaotropic substance. In addition, the chaotropic substance is corrosive and toxic. Therefore, from this respect, if it is possible to not use the chaotropic substance, such is very advantageous for a worker on safety of working operation. The chaotropic substance used herein is urea, guanidine salt, sodium isocyanate, sodium iodide, potassium iodide, and the like.

Conventionally, in the washing step in the separation and purification method of nucleic acid, the washing solution has high wettability to a container such as a cartridge, and consequently, the washing solution often remains in the container, resulting in inclusion of the washing solution in the recovering step subsequent to the washing step. This is a cause of, for example, a lowering of purity of nucleic acid or a lowering of reactivity in the next step. For this reason, when adsorption and desorption of nucleic acid are conducted using the container such as a cartridge, it is important that the residual washing solution does not remain in the cartridge such that a solution used in adsorbing and washing, particularly a washing solution, does not affect the next step.

Accordingly, to prevent that the washing solution in the washing step is mixed with the recovering solution in the next step, and to minimize the amount of the residual washing solution in the cartridge, the washing solution has a surface tension of preferably less than 0.035 J/m². When the washing solution has low surface tension, wettability between the washing solution and the cartridge is improved, and as a result, the amount of the residual solution can be suppressed.

To increase the washing efficiency, the proportion of waster in the washing solution can be increased. In this case, however, the surface tension of the washing solution rises, and the amount of residual solution increases. Where the surface tension of the washing solution is 0.035 J/m² or higher, the amount of the residual solution can be suppressed by increasing water repellency of the cartridge. By increasing water repellency of the cartridge, droplets are formed, and the droplets flow down, whereby the amount of the residual solution can be suppressed. A method for increasing the water repellency is, for example, a method of applying a water repellent to the surface of the cartridge, or a method of incorporating a water repellent such as a silicone when molding a cartridge, although not limited thereto.

The amount of the washing solution in the washing step is preferably 2 μl/mm² or less. When the amount of the washing solution is large, the washing effect is improved. However, when the amount is 200 μl/mm² or less, operating property can be maintained, and flow out of the sample can be suppressed, which is preferable.

In the washing step, the flow rate in the case of passing the washing solution through the solid phase is preferably from 2 to 1,500 μl/sec, more preferably from 5 to 700 μl/sec, per unit area (cm²) of the membrane. Where the passing rate is decreased and much time is taken, a sufficient washing will be performed. However, by setting the flow rate to the above range, the separation and purification operation of nucleic acid can quickly be conducted without lowering the washing efficiency, and this is preferable.

In the washing step, the temperature of the washing solution is preferably from 4 to 70° C., and more preferably room temperature. Further, in the washing step, stirring by mechanical vibration or ultrasonic wave can be applied to the cartridge for separation and purification of nucleic acid simultaneously with the washing step. Washing can also be performed by conducting a centrifuge.

Prior to or during the washing step, when the desired nucleic acid to be recovered is DNA, RNA can previously be degraded by contacting an RNA degrading enzyme solution with the solid phase. When the desired nucleic acid is RNA, DNA can previously be degraded by contacting a DNA degrading enzyme solution with the solid phase. In either case, it is important to subsequently remove the RNA degrading enzyme or DNA degrading enzyme from the solid phase by washing the solid phase using the washing solution.

### Recovery

The solid phase after washing is then contacted with a solution (recovering solution) capable of desorbing nucleic acid adsorbed on the solid phase. The solution contains the desired nucleic acid. Therefore, the solution is recovered, and then provided to the subsequent operation such as amplification of nucleic acid by PCR (polymerase chain reaction).

Volume of the recovering solution can be adjusted to volume of the sample solution containing nucleic acid prepared from a sample, and then desorption of nucleic acid can be conducted. The amount of the recovering solution containing nucleic acid separated and purified depends on the amount of the sample used. The amount of the recovering solution generally employed is from several ten to several hundred μl. However, when the amount of the sample is very slight, or when a large amount of nucleic acid is desired to separate and purify, the amount of the recovering solution can vary in a range of from 1 μl to several ten ml.

The recovering solution can preferably use purified distilled water, Tris/EDTA buffer, and the like. The recovering solution has pH of preferably from 2 to 11, and more preferably from 5 to 9. In particular, ionic strength and salt concentration advantageously affect elution of the adsorbed nucleic acid. The recovering solution has the ionic strength of preferably 290 mmol/liter or lower, and further has a salt concentration of 90 mmol/liter or lower. In this case, the salt may be an alkali metal salt. By setting the recovering solution to have the above properties, the recovery of nucleic acid can be improved, and as a result, a large amount of nucleic acid can be recovered.

By reducing the volume of the recovering solution as compared with the volume of the initial sample solution containing nucleic acid, the recovering solution containing concentrated nucleic acid can be obtained. Ratio of (volume of recovering solution) to (volume of sample solution) is preferably from 1:100 to 99:100, and more preferably 1:10 to 9:10. By this range of ratio, nucleic acid can be concentrated in a simple manner without operation for concentration in the step after separation and purification of nucleic acid. Thus, a method of obtaining a nucleic acid solution in which the nucleic acid is further concentrated by the above methods as compared with the sample can be provided.

The number of injection of the recovering solution is not limited, and may be one, or two or more. In general, where nucleic acid is separated and purified quickly and simply, a single recovery is conducted. On the other hand, where a large amount of nucleic acid is recovered, the recovering solution may be separately injected several times.

In the recovering step, a stabilizer can be added to the recovering solution of nucleic acid in order to prevent degradation of nucleic acid recovered. Examples of the stabilizer that can be added include an antibacterial agent, an antifungal agent, and a nucleic acid degradation inhibitor. The nucleic acid degradation inhibitor is an inhibitor of nucleic acid degrading enzyme, and is specifically EDTA. As other recovery embodiment, the stabilizer can previously be added to a recovery container. Further, unnecessary DNA such as chromosome genome DNA can be degraded by adding a specific DNA degrading enzyme solution to the nucleic acid solution recovered.

The method according to the present invention can apply to the case of separating and purifying plasmid DNA, and can also preferably apply to the case of separating and purifying phagimide DNA in a similar manner. Unit for Separation and Purification of Nucleic Acid and Cartridge for Separation and Purification of Nucleic Acid

In the present invention, it is preferable to use a unit for separation and purification of nucleic acid, comprising (a) a solid phase, (b) a container having at least two openings, which receives the solid phase, and (c) a pressure difference-generating apparatus joined to one opening of the container.

The unit for separation and purification of nucleic acid is described below.

A material for the container is not particularly limited so long as it can receive the solid phase, and can be provided with at least two openings. Plastics are preferably used from ease of production. For example, transparent or opaque resins such as a polystyrene, a polymethacylic ester, a polyethylene, a polypropylene, a polyester, nylon or a polycarbonate are preferably used as the plastics.

The container is provided with a solid phase-receiving portion, and the solid phase can be received in the receiving portion. The solid phase does not go out of the receiving portion at the time of suction and discharge of the sample solution or the like, and a pressure difference-generating apparatus such as a syringe can be joined to the opening. For such a container, it is preferable that the container is divided into two portions at the beginning, and after receiving the solid phase, the two portions can be united. Further, to avoid that the solid phase goes out of the receiving portion, a mesh prepared from a material which does not contaminate nucleic acid can be placed on the upper and lower sides of the solid phase.

Shape of the solid phase received in the container is not particularly limited, and the solid phase may have any shape of a circle, a square, a rectangle, an ellipse, a cylindrical shape where a membrane, a wound shape where a membrane, beads the surface which being coated with an organic polymer having a hydroxyl group, and the like. Highly symmetrical shapes such as a circle, a square, a cylindrical shape or a wound shape, and beads are preferably used from the standpoint of suitability of production.

The container is generally produced in a state that a body receiving the sold phase and a lid are separated, and each of the body and the lid is provided with at least one opening. The opening is used as an inlet and an outlet of a sample solution containing nucleic acid, a washing solution, and a solution capable of desorbing nucleic acid adsorbed on the solid phase (hereinafter all referred to as a “sample solution and the like” for simplicity). The opening is connected to the pressure difference-generating apparatus capable of making the inside of the container be reduced pressure state or pressurized state. Shape of the body is not particularly limited. However, in order that the production is easy and the sample solution and the like are easily diffused over the entire surface of the solid phase, the body preferably has a cross-section of a circle. The cross-section of a quadrangle is also preferable to prevent cut pieces of the solid phase from generation.

The lid is required to join to the body so as to make the inside of the container be reduced pressure state or pressurized state by the pressure difference-generating apparatus. However, any joining method can be selected so long as such a state can be achieved. Examples of the joining method include use of an adhesive, screwing, fitting, fixing with a screw, and fusion bonding with ultrasonic heating.

Inner volume of the container is determined by only the amount of the sample solution to be treated, but is generally indicated by the volume of the solid phase received. Specifically, the inner volume is preferably a volume that can receive about 1 to 6 solid phases each having a thickness of about 1 mm or less (for example, about 50 to 500 μm) and a diameter of from about 2 to 20 mm.

It is preferably that the edge portion of the solid phase in the container is closely contacted with an inner wall face of the container in an extent such that the sample solution and the like do not pass through the space between the solid phase and the inner wall.

The under portion of the solid phase facing the opening used as the inlet of the sample solution and the like is constructed such that the solid phase does not closely contact with the inner wall of the container to provide a space, so that the sample solution and the like diffuse over the entire surface of the solid phase uniformly as possible.

A member having a perforation (hole) at nearly the center thereof is preferably provided on the upper of the solid phase facing the opening joined to the pressure difference-generating apparatus. This member has the function to press down the solid phase and also the effect to discharge the sample solution and the like with good efficiency. The member preferably has a shape having a slanting surface, such as a funnel shape or a bowl shape, so as to concentrate the solution in the central hole. A size of the hole, an angle of the slanting surface and a thickness of the member can appropriately be determined by one skilled in the art, taking into consideration an amount of the sample solution and the like to be treated, and a size of the container receiving the solid phase. A space for storing the sample solution and the like overflowed, thereby preventing the same from being sucked in the pressure difference-generating apparatus is preferably provided between the member and the opening. A size of this space can appropriately be determined by one skilled in the art. To efficiently collect nucleic acid, it is preferable to suck the sample solution containing nucleic acid, in at least an amount such that the entire solid phase sufficiently dips therein.

To prevent that the sample solution and the like concentrate at only the portion just below the opening under sucking operation, whereby the sample solution and the like can pass through the inside of the solid phase relatively uniformly, a space is also preferably provided between the solid phase and the member. To achieve this construction, a plurality of projections is provided toward the solid phase from the member. A size and the number of the projection can be determined by one skilled in the art. However, it is preferable to maintain the opening area of the solid phase large as possible while holding the space.

Where at least three openings are provided on the container, needless to say it is necessary to temporarily seal the superfluous openings so as to enable suction and discharge of the solution due to pressure-reducing and pressuring operations.

The pressure difference-generating apparatus first functions to suck the sample solution containing nucleic acid by reducing pressure in the container having the solid phase received therein. The pressure difference-generating apparatus includes a pump capable of performing suction and pressuring, such as a syringe, a pipette and a perista pump. Of those, the syringe is suitable for manual operation, and the pump is suitable for automatic operation. The pipette has the advantage that it can easily be operated with one hand. Preferably, the pressure difference-generating apparatus is detachably joined to one opening of the container.

The method for separating and purifying nucleic acid using the above-described unit for separation and purification of nucleic acid is described below.

Preferably, in the method for separating and purifying nucleic acid according to the present invention, adsorption and desorption of nucleic acid can be conducted using the cartridge for separation and purification of nucleic acid in which the solid phase is received in the container having at least two openings.

More preferably, adsorption and desorption of nucleic acid can be conducted using the cartridge for separation and purification of nucleic acid, comprising (a) the solid phase, (b) a container having at least two openings, which receives the solid phase, and (c) a pressure difference-generating apparatus joined to one opening of the container.

In this case, a first embodiment of the method for separating and purifying nucleic acid according to the present invention can include the following steps.

(a) a step of adding a dispersing solution to a sample (bacteria or cells);

(b) a step of adding an alkali solution to the solution obtained in (a) above to dissolve the sample therein;

(c) a step of adding a neutralizing solution to the solution obtained in (b) above to precipitate unnecessary materials other than the desired nucleic acid;

(d) a step of adding a lysis solution to a supernatant solution of the precipitate obtained in (c) above;

(e) a step of adding a water-soluble organic solvent to the solution obtained in (d) above to prepare a sample solution (a solution for adsorbing nucleic acid on a solid phase);

(f) a step of inserting one opening of a unit for separation and purification of nucleic acid in the sample solution;

(g) a step of sucking the solution for adsorbing nucleic acid on a solid phase by reducing pressure in the container using a pressure difference-generating apparatus joined to other opening of the unit for separation and purification of nucleic acid, thereby contacting the solution with the solid phase;

(h) a step of discharging the sucked solution for adsorbing nucleic acid on a solid phase out of the container by increasing pressure in the container using the pressure difference-generating apparatus joined to other opening of the unit for separation and purification of nucleic acid;

(i) a step of inserting one opening of the unit for separation and purification of nucleic acid in a washing solution;

(j) a step of sucking the washing solution by reducing pressure in the container using the pressure difference-generating apparatus joined to other opening of the unit for separation and purification of nucleic acid, thereby contacting the washing solution with the solid phase;

(k) a step of discharging the sucked washing solution out of the container by increasing pressure in the container using the pressure difference-generating apparatus joined to other opening of the unit for separation and purification of nucleic acid;

(l) a step of inserting one opening of unit for separation and purification of nucleic acid in a solution (recovering solution) capable of desorbing nucleic acid adsorbed on the solid phase;

(m) a step of sucking the solution capable of desorbing nucleic acid adsorbed on the solid phase by reducing pressure in the container using the pressure difference-generating apparatus joined to other opening of the unit for separation and purification of nucleic acid, thereby contacting the solution with the solid phase; and

(n) a step of discharging the solution capable of desorbing nucleic acid adsorbed on the solid phase out of the container by increasing pressure in the container using the pressure difference-generating apparatus joined to other opening of the unit for separation and purification of nucleic acid.

In the steps (g), (j) and (m), it is preferable to suck the solution in an amount substantially contacting with the entire solid phase. However, where the solution is sucked in the pressure difference-generating apparatus, the apparatus is contaminated with the solution. Therefore, the amount of solution sucked is controlled to an appropriate amount. After sucking an appropriate amount of the solution, the inside of the container is pressurized using the pressure difference-generating apparatus to discharge the sucked solution. Interval is not required until this operation, and the solution may be discharge immediately after suction.

A second embodiment of the method for separating and purifying nucleic acid according to the present invention can include the following steps.

(a) a step of adding a dispersing solution to a sample (bacteria or cells);

(b) a step of adding an alkali solution to the solution obtained in (a) above to dissolve the sample therein;

(c) a step of adding a neutralizing solution to the solution obtained in (b) above to precipitate unnecessary materials other than the desired nucleic acid;

(d) a step of adding a lysis solution to a supernatant solution of the precipitate obtained in (c) above;

(e) a step of adding a water-soluble organic solvent to the solution obtained in (d) above to prepare a sample solution (a solution for adsorbing nucleic acid on a solid phase);

(f) a step of injecting the sample solution in one opening of a unit for separation and purification of nucleic acid;

(g) a step of discharging the injected solution for adsorbing nucleic acid on a solid phase out of other opening by increasing pressure in the container using a pressure difference-generating apparatus joined to the one opening of the unit for separation and purification of nucleic acid;

(h) a step of injecting a washing solution in the one opening of the unit for separation and purification of nucleic acid;

(i) a step of discharging the injected washing solution out of the other opening by increasing pressure in the container using the pressure difference-generating apparatus joined to the one opening of the unit for separation and purification of nucleic acid;

(j) a step of injecting a solution (recovering solution) capable of desorbing nucleic acid adsorbed on the solid phase in the one opening of the unit for separation and purification of nucleic acid; and

(k) a step of discharging the injected solution capable of desorbing nucleic acid adsorbed on the solid phase out of the other opening by increasing pressure in the container using the pressure difference-generating apparatus joined to the one opening of the unit for separation and purification of nucleic acid, thereby desorbing nucleic acid adsorbed on the solid phase and discharging the nucleic acid out of the container.

In the above steps, there is no limitation in injecting the sample solution in the container, and experimental instruments such as a pipette or a spoil are preferably used. Those instruments are more preferably nuclease-free or hydrogen-free.

A method for mixing the sample and each solution is not particularly limited. For example, mixing is preferably conducted at from 30 to 3,000 rpm for from 1 second to 3 minutes using a stirring device. By this mixing, the yield of nucleic acid separated and purified can be increased. Alternatively, it is preferable to mix by conducting rollover mixing 5 to 30 times. Further, it can be mixed by conducting pipetting operation from 10 to 50 times.

A kit comprising (i) a cartridge for separation and purification of nucleic acid, (ii) a plurality of enzymes, (iii) a pre-treating solution containing at lest one of a chaotropic salt, a surfactant, a defoaming agent, a nucleic acid stabilizer, a buffer, an acid and an alkali agent, and a water-soluble organic solvent, (iv) a washing solution, and (v) a reagent of recovering solution can be prepared and used.

Example of an automatic apparatus which automatically conducts the step of separating and purifying nucleic acid from the sample containing nucleic acid using a cartridge for separation and purification of nucleic acid, having the solid phase received in a container having at least tow openings, and a pressure difference-generating apparatus is described below, but the automatic apparatus is not limited to this.

The automatic apparatus is an apparatus for separation and purification of nucleic acid, which automatically conducts separation and purification actions as follows. A cartridge for separation and purification of nucleic acid, having the solid phase received therein, wherein a solution can pass through the inside of the cartridge, is used. A solution for adsorbing nucleic acid on the solid phase (sample solution) is injected in the cartridge for separation and purification of nucleic acid, followed by pressurizing, thereby adsorbing nucleic acid in the sample solution on the solid phase. A washing solution is injected in the cartridge for separation and purification of nucleic acid to pressure, thereby removing impurities. A recovering solution is injected in the cartridge for separation and purification of nucleic acid to desorb nucleic acid adsorbed on the solid phase, and the desorbed nucleic acid is recovered together with the recovering solution. Thus, the automatic apparatus comprises a mounting mechanism holding the cartridge for separation and purification of nucleic acid, a waste solution container which receives the sample solution and the washing solution, and a recovering container which receives the recovering solution containing nucleic acid; a pressured air supplying mechanism which introduces pressurized air into the cartridge for separation and purification of nucleic acid; and an injection mechanism which separately injects the washing water and the recovering solution in the cartridge for separation and purification of nucleic acid.

The mounting mechanism preferably comprises a stand mounted on the apparatus body, a cartridge holder that holds the cartridge for separation and purification of nucleic acid up and down movably supported by the stand, the waste solution container, the position of which is exchangeable to the cartridge for separation and purification of nucleic acid at a lower portion of the cartridge, and a container holder which holds the recovering container.

The pressurized air supplying mechanism preferably comprises an air nozzle which ejects pressurized air from the lower portion thereof, a pressurizing head which supports the air nozzle and makes the air nozzle move up and down to the cartridge for separation and purification of nucleic acid held by the cartridge holder, and a means for determining the position of the cartridge for separation and purification of nucleic acid in a rack of the mounting mechanism provided on the pressurizing head.

The injection mechanism preferably comprises a washing solution injecting nozzle which injects the washing solution, a recovering solution injecting nozzle which injects the recovering solution, a nozzle movable carriage which holds the washing solution injecting nozzle and the recovering solution injecting nozzle and is movable in turn on the cartridge for separation and purification of nucleic acid held by the mounting mechanism, a washing solution supplying pump which sucks the washing solution from a washing solution bottle having the washing solution received therein and supplies the washing solution to the washing solution injecting nozzle, and a recovering solution supplying pump which sucks the recovering solution from the recovering solution bottle having the recovering solution received therein and supplies the recovering solution to the recovering solution injecting nozzle.

According to the above automatic apparatus, it is provided with the mounting mechanism which holds the cartridge for separation and purification of nucleic acid, the waste solution container and the recovering container, the pressured air supplying mechanism which introduces pressurized air into the cartridge for separation and purification of nucleic acid, and the injection mechanism which separately injects the washing solution and the recovering solution in the cartridge for separation and purification of nucleic acid. Further, the apparatus automatically performs each step in the method for separating and purifying nucleic acid of injecting under pressure a solution for adsorbing nucleic acid on a solid phase in the cartridge for separation and purification of nucleic acid equipped with the solid phase member to adsorb the nucleic acid on the solid phase member, injecting a washing solution to wash and discharge impurities, and injecting a recovering solution to separate and recover the nucleic acid adsorbed on the solid phase member. Thus, a mechanism that can automatically conduct separation and purification of nucleic acid in a sample solution for a short period of time with good efficiency can be constituted in a compact manner.

When the mounting mechanism comprises the stand, the up and down movable cartridge holder that holds the cartridge for separation and purification of nucleic acid, and a container holder which holds the waste solution container and the recovering container in an exchangeable manner, exchange of the cartridge for separation and purification of nucleic acid, and each or a set of the waste solution container and the recovering container can easily be conducted.

When the pressurized air supplying mechanism comprises the air nozzle, the pressurizing head which moves the air nozzle up and down, and the position determining means for determining the position of the cartridge for separation and purification of nucleic acid, pressurized air can securely be supplied in a simple mechanism.

When the injection mechanism comprises the washing solution injecting nozzle, the recovering solution injecting nozzle, the nozzle movable carriage which is movable in turn on the cartridge for separation and purification of nucleic acid, and the washing solution supplying pump which sucks the washing solution from a washing solution bottle and supplies the washing solution to the washing solution injecting nozzle, and the recovering solution supplying pump which sucks the recovering solution from the recovering solution bottle and supplies the recovering solution to the recovering solution injecting nozzle, injection of the washing solution and the recovering solution can successively be conducted in a simple mechanism.

The present invention is described in more detail by reference to the following examples, but it should be understood that the invention is not construed as being limited thereto.

### EXAMPLE

### Example 1

### (1) Preparation of Cartridge for Separation and Purification of Nucleic Acid

A cartridge for separation and purification of nucleic acid having an inner diameter of 7 mm and having a portion receiving a porous membrane as a solid phase was prepared by a high impact polystyrene.

(2) A porous membrane (pore size: 2.5 μm, diameter: 7 mm, thickness: 100 μm, degree of saponification: 95%) obtained by saponification of a porous membrane comprising triacetycellulose was used as the porous membrane, and was received in the solid phase receiving portion of the cartridge for separation and purification of nucleic acid prepared (1) above.

### (3) Preparation of Dispersing Solution, Alkali Solution, Neutralizing Solution, Lysis Solution, Washing Solution and Recovering Solution

Dispersing solution, alkali solution, neutralizing solution, lysis solution, washing solution and recovering solution for separation and purification of plasmid DNA each having the following formulation were prepared. Dispersing solution (for separation and purification of plasmid DNA)

| | | |
|---|---|---|
| 1 mol/liter trishydrochloric salt (a product of Wako | 26 | g |
| Pure Chemical Industries, Ltd.) | | |
| 0.5 mol/liter EDTA (a product of Wako Pure Chemical | 11 | g |
| Industries, Ltd.) | | |
| Distilled water | 465 | g |
| Alkali solution (for separation and preparation of | | |
| plasmid DNA) | | |
| 1 mol/liter NaOH (a product of Wako Pure Chemical | 104 | g |
| Industries, Ltd.) | | |
| 10 wt % SDS (a product of Wako Pure Chemical Industries, | 50 | g |
| Ltd.) | | |
| Distilled water | 350 | g |
| Neutralizing solution (for separation and purification of | | |
| plasmid DNA) | | |
| Potassium acetate (a product of Wako Pure Chemical | 147 | g |
| Industries, Ltd.) | | |
| Acetic acid (a product of Wako Pure Chemical | 68 | g |
| Industries, Ltd.) | | |
| Distilled water | 356 | g |
| Lysis solution (for separation and purification of | | |
| plasmid DNA) | | |
| Tween-20 (a product of ICN Pharmaceutical) | 39 | g |
| BiS-Tris (a product of Wako Pure Chemical Industries, | 3.4 | g |
| Ltd.) | | |
| Ethanol (99.5%) (a product of Wako Pure Chemical | 344 | ml |
| Industries, Ltd.) | | |
| Distilled water | 104 | ml |
| Washing solution (for separation and purification of | | |
| plasmid DNA) | | |
| 1 mol/liter trishydrochloric acid (a product of Wako | 5.6 | g |
| Pure Chemical Industries, Ltd.) | | |
| Ethanol (99.5%) (a product of Wako Pure Chemical | 400 | ml |
| Industries, Ltd.) | | |
| Distilled water | 94 | g |
| Recovering solution (for separation and purification of | | |
| plasmid DNA) | | |
| 1 mol/liter trishydrochloric acid (a product of Wako | 5.2 | g |
| Pure Chemical Industries, Ltd.) | | |
| Distilled water | 494 | g |

(4) Extraction of Plasmid DNA from E. coli pBluescript IISK(−)/DH5α
(i) Preparation of E. coli pBluescript IISK(−)/DH5a

E. coli DH5α transformant (referred to as “pBs II(−)/DH5α”) transformed with plasmid pBluescript IISK(−) (a product of Stratagene) was inoculated in 100 ml of Luria-Bertani broth (10 g/liter tryptone, 5 g/liter yeast extract, 5 g/liter sodium chloride (pH:7.5)) containing 100 μg/ml of ampicillin, and cultivated at a cultivation temperature of 37° C. and a shaking speed of 220 min⁻¹ for 15 hours. After the cultivation, the culture solution was separately injected in a 1.5 ml nuclease-free and hydrogen-free microtube (platinum tube, a product of BM Equipment Co.) with 1.0 ml portions. The culture solution was centrifuged with a high speed refrigerated microcentrifuge (trade name: MX-300, a product of Tomy Seiko Co.) at 60000× g for 15 minutes. The supernatant solution was removed to obtain a biomass. This was used as a material for extraction.

### (ii) Separation and Purification of Plasmid DNA

Each of 3 μl of a 10 mg/ml RNase A (a product of Wako Pure Chemical Industries, Ltd.) solution, and 1 μl of a 1 mg/ml RNase T1 (a product of Sigma Co.) solution was added to 100 μl of the dispersing solution prepared in (3) above containing the biomass prepared in (i) above, and the resulting solution was stirred by vortex at room temperature for 15 seconds to securely disperse the biomass. 100 μl of the alkali solution prepared in (3) above was then added to the solution, and rollover mixing was conducted five times to perform bactriolysis of the biomass. 140 μm of the neutralizing solution prepared in (3) above was then added to the solution, and rollover mixing was conducted 5 times to neutralize a sample solution. The precipitate residue was centrifuged with a high speed refrigerated microcentrifuge (trade name: MX-300, a product of Tomy Seiko Co.) at 18000×g for 10 minutes to recover 330 μl of a supernatant solution. 320 μl of the lysis solution prepared in (3) above was previously added to a fresh 1.5 ml nuclease-free and hydrogen-free microtube (platinum tube, a product of BM Equipment Co.), and the supernatant solution obtained above was added to the container. Stirring was conducted by vortex for 30 seconds to obtain a sample solution.

The respective solution was separately injected in one opening of the cartridge for separation and purification of nucleic acid having the porous membrane as a solid phase prepared in (2) above, and a pressure difference-generating apparatus (tubing pump) was joined to the one opening. The inside of the cartridge for separation and purification of nucleic acid was made a pressurized state (80 kPa), and the injected solution was passed through the solid phase of the porous membrane, thereby contacting the solution with the solid phase. The solution was discharged from other opening of the cartridge for separation and purification of nucleic acid. The washing solution prepared in (3) above was injected in the one opening of the cartridge for separation and purification of nucleic acid, and a tubing pump was joined to the one opening. The inside of the cartridge for separation and purification of nucleic acid was made a pressurized state (80 kPa), and the injected washing solution was passed through the solid phase of the porous membrane, and then discharged from other opening. The recovering solution prepared in (3) above was injected in the one opening of the cartridge for separation and purification of nucleic acid, and a tubing pump was joined to the one opening of the cartridge for separation and purification of nucleic acid. The inside of the cartridge for separation and purification of nucleic acid was made a pressurized state (80 kPa), and the injected recovering solution was passed through the solid phase of the porous membrane, and then discharged from other opening. The solution was then recovered. Time required for the nucleic acid separation and purification operation (from injection of the sample solution containing nucleic acid to recovery thereof) was 6 minutes.

The following experiment was conducted as the comparative example.

The biomass prepared in (i) above was subjected to separation and purification operation using QIAGEN QIAprep mini according to protocol of the kit to obtain a recovering solution containing nucleic acid.

In (ii) above, only RNase A (a product of Wako Pure Chemical Industries, Ltd) was added to the dispersing solution, and the resulting solution was subjected to separation and purification operation to obtain a recovering solution containing nucleic acid.

In (ii), only RNase T1 (a product of Sigma Co.) was added to the dispersing solution, and the resulting solution was subjected to separation and purification operation to obtain a recovering solution containing nucleic acid.

### (5) Quantitative Determination of Amount of Nucleic Acid Recovered

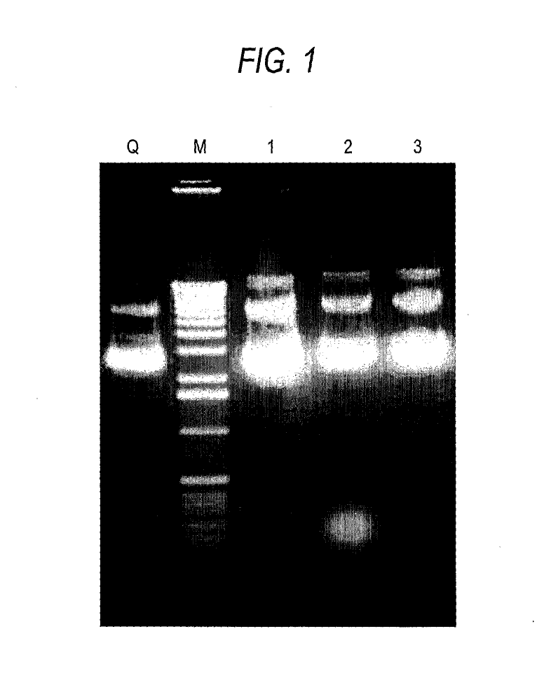

With respect to the respective recovering solution recovered in the above Examples, the results of electrophoresis of DNA are shown in the attached FIG. 1. Conditions of electrophoresis in this case are as follows.

1% agarose/1xTAW buffer, EtBr dyeing 35 minutes migration Sample 2 μl+Loading buffer 1 μl→3 μl whole amount apply

Absorbance at 260 nm is shown in the Table 1 below.

TABLE 1

| | Q | 1 | 2 | 3 |
|---|---|---|---|---|
| Absorbance | 6.9 | 10 | 8.5 | 6.6 |
| | Comparative | Comparative | Comparative | Invention |
| | Example | Example | Example | |

The symbols in the above Table are as follows.

Q: Extraction with QIAGEN QIAprep mini

1: Adding only RNase A (a product of Wako Pure Chemical Industries, Ltd.) to a dispersing solution

2: Adding only RNase T1 (a product of Sigma Co.) to a dispersing solution

3. Adding RNase A (a product of Wako Pure Chemical Industries, Ltd.)+RNase T1 (a product of Sigma Co.) to a dispersing solution

As is apparent from the electrophoresis in the FIG. 1 and the results shown in the Table 1, the invention example (lane 3) can purify plasmid DNA having less contamination of RNA originated from a host, and can obtain genome DNA having apparently high purity as compared with the sample (lane Q) extracted with QIAGEN QIAprep mini as the Comparative Example, and the conventional methods (lanes 1 and 2) using a single enzyme. In other words, the method according to the present invention exhibits excellent separation performance and good washing efficiency, and as a result, plasmid DNA can be obtained quickly with the above-described period of time in high yield and high purity.

### INDUSTRIAL APPLICABILITY

The method of the present invention makes it possible to separate high purity plasmid DNA from a sample solution containing nucleic acid prepared from bacteria or cells with good efficiency.

The entire disclosure of each and every foreign patent application from which the benefit of foreign priority has been claimed in the present application is incorporated herein by reference, as if fully set forth.

1. A method for separating and purifying nucleic acid, the method comprising:
(1) a step of contacting a sample solution containing nucleic acid with a solid phase to adsorb the nucleic acid on the solid phase;
(2) a step of contacting a washing solution with the solid phase to wash the solid phase in a state that the nucleic acid is adsorbed on the solid phase; and
(3) a step of contacting a recovering solution with the solid phase to desorb the nucleic acid from the solid phase,
wherein the sample solution is prepared by including a step of adding at least two enzymes.

2. The method for separating and purifying nucleic acid according to claim 1,
wherein the at least two enzymes comprise at least one nucleic acid degrading enzyme.

3. The method for separating and purifying nucleic acid according to claim 2,
wherein the at least one nucleic acid degrading enzyme is at least one RNA degrading enzyme.

4. The method for separating and purifying nucleic acid according to claim 3, wherein the at least one RNA degrading enzyme comprises RNase T1 and RNase A.

5. The method for separating and purifying nucleic acid according to claim 1,
wherein the sample solution is prepared by adding a pre-treating solution containing at least one selected from a chaotropic salt, a surfactant, a defoaming agent, a nucleic acid stabilizer, a buffer, an acid and an alkali agent, and further adding a water-soluble organic solvent thereto.

6. The method for separating and purifying nucleic acid according to claim 1,
wherein the solid phase is a membrane-shaped solid phase.

7. The method for separating and purifying nucleic acid according to claim 1,
wherein the water-soluble organic solvent contains at least one selected from methanol, ethanol, propanol and its isomer and butanol and its isomer.

8. The method for separating and purifying nucleic acid according to claim 1,
wherein the solid phase contains silica or its derivative, diatomaceous earth or alumina.

9. The method for separating and purifying nucleic acid according to claim 1,
wherein the solid phase contains an organic polymer.

10. The method for separating and purifying nucleic acid according to claim 9,
wherein the solid phase contains at least one selected from Teflon®, a polyester, a polyether sulfone, a polycarbonate, a polyacrylate copolymer, a polyurethane, a polybenzimidazole, a polyolefin, a polyvinyl chloride and a polyvinylidene fluoride.

11. The method for separating and purifying nucleic acid according to claim 9,
wherein the solid phase contains nylon having positive or negative charges.

12. The method for separating and purifying nucleic acid according to claim 9,
wherein the organic polymer has a polysaccharide structure.

13. The method for separating and purifying nucleic acid according to claim 9,
wherein the organic polymer contains at least one selected from cellulose, cellulose mixed ester, cellulose nitrate, cellulose acetate and nitrocellulose.

14. An apparatus for automatically conducting the steps in a method for separating and purifying nucleic acid according to claim 1.

15. A kit for conducting a method for separating and purifying nucleic acid according to claim 1, the kit comprising:
(i) a cartridge for separation and purification of nucleic acid;
(ii) at least two enzymes;
(iii) a pre-treating solution containing at least one selected from a chaotropic salt, a surfactant, a deforming agent, a nucleic acid stabilizer, a buffer, an acid and an alkali agent, and a water-soluble organic solvent;
(iv) a washing solution; and
(v) a reagent of a recovering solution.
